(19) United States
(12) Patent Application Publication
(10) Pub. No.: US 2005/0255475 A1
(43) Pub. Date: Nov. 17, 2005

(54) Energy transfer dyes, terminators and use thereof

(76) Inventors: Shiv Kumar, Belle Mead, NJ (US); Chung-Yuan Chen, Piscataway, NJ (US); Sudhakar Rao, Belle Mead, NJ (US)

Correspondence Address: AMERSHAM BIOSCIENCES;PATENT DEPARTMENT, 800 CENTENNIAL AVENUE, PISCATAWAY, NJ, 08855, US

(21) Appl. No.: 10/528,863

(22) PCT Filed: Sep. 25, 2003
(86) PCT No.: PCT/US03/30360

§ 371 Date: Mar. 23, 2005

Related U.S. Application Data
(60) Provisional application No. 60/413,517, filed on Sep. 25, 2002.

(51) Int. Cl. C12Q 1/68; C07D 473/02

(52) U.S. Cl. 435/6; 544/244; 544/230

(57) ABSTRACT

The present invention relates to a novel set of four fluorescently labeled dye terminators. Two of them are single dye labeled terminators and the other two dye terminators are based on fluorescent resonance energy transfer (FRET). The FRET dye terminators are generated from the 4′,5′-bis-aminomethylfluorescein. Of the two amino groups of the donor dye, 4′,5′-bis-aminomethylfluorescein, one amino group is used to attach the acceptor dye, and the other amino group is used to attach the dideoxynucleoside-5′-triphosphate. Their preparation, energy transfer efficiency, and use as labels, specifically, in DNA sequencing reactions is disclosed.

### BACKGROUND OF THE INVENTION

1. Field of the Invention

The present invention relates to energy transfer dyes and fluorescently labeled dye terminators, their preparation and use as labels in DNA sequencing.

This application claims priority benefit under Title 35 §119(e) from U.S. provisional patent application No. 60/413,517, filed Sep. 25, 2002.

The currently accepted practice of high throughput gene sequencing employs, as a general rule, four differently labeled Energy Transfer (ET) dye terminators based on Forster Resonance Energy Transfer (FRET) mechanism to read out the sequence by exciting at one excitation wavelength of the donor and measuring the emissions at the wavelength of the four acceptors conjugated to the four individual nucleic acid bases.

However, the currently available ET terminator sets as described suffer from low brightness. This low brightness is due to the inefficiencies in the transfer of the energy absorbed by the donor to the acceptors and re-emitance at the emission wavelength of the acceptor. This inefficiency arises because of the structural linkages used to join the donor and the acceptors as well as the bases together to form the dye terminators are less than optimal.

A large number of fluorescent dyes have been recently developed for labeling and detecting components in biological samples. Generally, these fluorescent dyes must have high extinction coefficient and quantum yield so a low detection limit can be achieved.

One class of the dyes which has been developed to give large and different Stokes shifts, based on Foster Resonance Energy Transfer (FRET) mechanism and used in the simultaneous detection of differently labeled sample in a mixture, is the ET (Energy Transfer) dyes. These ET dyes include a complex molecular structure consisting of a donor fluorophore and an acceptor fluorophore as well as a labeling function to allow its conjugation to biomolecules of interests. Upon excitation of the donor fluorophore, the energy absorbed by the donor is transferred by the Forster Resonance Energy Transfer (FRET) mechanism to the acceptor fluorophore and causes it to fluoresce. With a single donor, different acceptors can be used to form a set of ET dyes so when the set is excited at one single donor frequency, various emissions can be observed depending on the choice of the acceptors. Upon quantification of these different emissions, the components of a mixture can readily be resolved when these dyes are conjugated to bio-molecules of interest. These ET dye sets constitute the backbone of current high throughput gene sequencing methodology.

2. Description of Related Art

Previously, a variety of combinations of bi-fluorophore dyes have been described. U.S. Pat. No. 5,688,648, entitled “Probes Labelled with Energy Transfer Coupled Dyes” Mathies et. al., U.S. Pat. No. 5,728,528, entitled “Universal spacer/energy transfer dyes, and U.S. Pat. No. 6,150,107, entitled “Methods of sequencing and detection using energy transfer labels with cyanine dyes as donor chromophores” which are incorporated herein by reference in its entirety, including any drawings, discloses sets of fluorescent labels carrying pairs of donor and acceptor dye molecules wherein the labels can be attached to nucleic acid backbone for sequencing. The nucleic acid bases or the basic sugar units are used as spacers to separate the donor and acceptor dyes. The optimum distance for efficient energy transfer from the donor dye to the acceptor dye was found to be ˜6-10 bases. Included is a method for identifying and detecting nucleic acids in a multi-nucleic acid mixture by using different fluorescent labels, wherein the fluorescent moieties are selected from families such as cyanine dyes and xanthenes. The fluorescent labels comprise pairs of fluorophores where one fluorophore donor has emission spectra, which overlaps the fluorophore acceptor's absorption so that there is energy transfer from the excited member to the other member of the pair.

U.S. Pat. No. 6,008,373, entitled “Fluorescent labeling complexes with large stokes shift formed by coupling together cyanine and other fluorochromes capable of resonance energy transfer” Waggoner et. al., which is incorporated herein by reference in it's entirety, including any drawings, discloses complexes comprising a first fluorochrome having first absorption and emission spectra and a second fluorochrome having second absorption and emission spectra. The linker groups between the fluorochromes are alkyl chains. The fluorescent nature of the dyes enables them to be of use in sequencing and nucleic acid detection.

U.S. Pat. No. 5,863,727, entitled “Energy transfer dyes with enhanced fluorescence” Lee et al., which is incorporated herein by reference in its entirety, discloses energy transfer dyes in which the donor and acceptor dyes are separated by a linker between the dyes. The preferred linker between the dyes is 4-aininomethylbenzoic acid (Nucleic Acids Research, 1997, 25(14), 2816-2822). The energy transfer terminators DNA sequencing kit based on this linker is commercially available from Applied Biosystems (Foster City, Calif.) and sold as Big Dye terminator kit.

PCT application WO 00/13026 entitled “Energy Transfer Dyes” Kumar et al., which is incorporated herein by reference in its entirety, including any figures and drawings, discloses energy transfer dyes, their preparation, and their use as labels in biological systems. The dyes are preferably in the form of cassettes, which enable their attachment to a variety of biological materials. The donor dye, acceptor dye and the dideoxynucleoside-5′-triphosphates are all attached to a trifunctional linker, which is based on aromatic aminoacids structure (Tetrahedron Letters, 2000, 41, 8867-8871). The energy transfer terminator kit based on these structures is sold by Amersham Biosciences, Piscataway, (N.J.) as DYEnamic ET terminator kit for DNA sequencing.

PCT application WO 01/19841 entitled “Charge-modified nucleic acids terminators” Kumar et al., which is incorporated herein by reference in its entirety, including any figures and drawings, discloses single and energy transfer dye labeled terminators with positive or negative charge(s) incorporated in the linker arm. These terminators are useful in generating DNA sequencing bands free of any ‘dye blobs’ which are formed by the degradation of dye labeled dideoxynucleoside-5′-triphosphates. The use of charge terminators allows these degradation products to move backward (positive charge terminators) or move ahead of sequence information (negative charge terminators, Finn et. al. Nucleic Acids Research, 2002, 30(13), 2877-2885, Finn et. al. Nucleic Acids Research, 2003, 31, 4769-4778.

However, the ET terminator sets as described in these patents suffer from low brightness. This low brightness is due to the inefficiencies in the transfer of the energy absorbed by the donor from the acceptors and its re-emission at the emission wavelength of the acceptor. This inefficiency arises because of the structural linkages used to join the donor and the acceptors as well as the bases together to form the dye terminators are less than optimal. Therefore, there remains a need for additional improvements in energy transfer dye construction for maximum brightness and their attachment to the biological molecules.

### SUMMARY OF THE INVENTION

The present invention, provides a novel set of four differently labeled dye terminators having much higher brightness than the currently available terminators. Two of the dye-terminators in this set are made up of single dyes and the other two with traditional ET dyes but with much better ET efficiency and other characteristics. The increase in brightness for the set of dye terminators of this invention and the corresponding improvement in signal to noise allow sequencing of a broader range of template amounts used. Furthermore, this new set of dye terminators provides electropherograms that exhibit significant improved peak uniformity and eliminate some of the sequence specific artifacts. The novel fluorophore/linker combination, in the form of 4′,5′ bis-aminomethyl fluorescein and its 5 or 6-carboxy substituted derivative, disclosed in this invention, also allows the constructions of trifluor (three chromophores) ET dyes. The trifluor dyes, having more than one donor and/or acceptor in addition to the labeling functionality, can be used to extend the possible range of acceptor emissions, upon a single wavelength excitation. This will result in more channels for monitoring and brighter emissions for sequencing reactions as well as other possible labeling applications. The 4′,5′-bis-aminomethyl fluorescein-5(6)-carboxylic acid skeleton also provide extra site for the attachment of positively or negatively charged moieties which could be used to synthesize fluorescein dyes of various charges and charged energy transfer dyes. The charged energy transfer dye terminators of this invention may be used for direct load ‘blob-free’ DNA sequencing.

The current invention provides energy transfer dyes and labeled nucleotides, which are brighter than the existing dye terminators and are substrate for DNA polymerases. The energy transfer dyes are based on the 4′,5′-bis-aminomethyl fluorescein structure. In this system, the acceptor dyes are attached directly to the amino group without the need of another linker to attach the acceptor dye. The second amino group of this structure is explored to attach the biological molecule of interest, such as nucleoside, nucleotide, oligonucleotide or other biological molecule of interest.

The current invention also provides single fluorescein structure based dyes or energy transfer dyes of various charges. The 4′,5′-bis-aminomethyl fluorescein or its 5(6)-carboxylic acid structure may be used to attach the positive or negative charged moieties. The charged dyes and terminators of this invention may also be used for ‘direct load’ DNA sequencing.

The current invention also provides a set of four terminators. The terminator set include two single dye (fluorescein (FAM), rhodamine 110 (R110) or rhodamine 6G (REG or R6G)) labeled dideoxynucleoside-5′-triphosphates and two energy transfer dye (fluorescein-tetramethylrhodamine (FAM-TAMRA) and fluorescein-rhodamine X (FAM-ROX) labeled dideoxynucleoside-5′-triphosphates. This terminator set is optimized for DNA sequencing. The labeled nucleotide terminators in the set are much brighter than the existing kits and gives more uniform bands. The method of their preparation and use in DNA sequencing is also disclosed in the present invention.

Disclosed are compositions and methods of making the energy transfer dyes of this invention and their attachment to the biological molecules of interest such as nucleosides, nucleotides (mono, di, or triphosphates) or oligonucleotides.

### BRIEF DESCRIPTION OF THE DRAWINGS

FIG. 1 presents a schematic for the synthesis of 4′,5′-bis-aminomethyl fluorescein and its 5-carboxylic acid.

FIG. 2 presents possible sites for the attachment of various charges and biological molecules on 4′,5′-bis-aminomethyl fluorescein and its 5-carboxylic acid structure.

FIG. 3 presents a schematic for the synthesis of energy transfer dyes and terminators based on 4′-aminomethyl-5-carboxylic acid structure.

FIG. 4 presents a schematic for the synthesis of energy transfer dyes and terminators based on 4′,5′-bis-aminomethyl fluorescein-5-carboxylic acid structure.

FIG. 5 presents a schematic for the structures of single dye labeled terminators and ET terminators of this invention.

FIG. 6 presents a schematic for the preferred dye terminator set: Structures of 2 single dye terminators and 2 ET terminators of this invention.

FIG. 7 presents normalized emission spectrum of the dye terminators optimized for DNA sequencing.

FIG. 8 presents a comparison of relative brightness of sequencing bands generated using dye terminators of this invention with the other commercially available terminators.

FIG. 9 presents uniformity of sequencing bands using terminators of this invention and their comparison with the existing commercial terminators.

FIG. 10 presents sequencing data of a DNA molecule using 2 single and 2 ET dye terminators described in this invention.

FIG. 11 presents sequencing band uniformity with different linker length FAM-ddGTP using various mutant DNA polymerases.

FIG. 12 present Sequencing band uniformity with different linker length REG-ddUTP using various mutant DNA polymerases.

FIG. 13 present Sequencing band uniformity with different linker length AMFAM-ROX-ddCTP using various mutant DNA polymerases.

FIG. 14 present Sequencing band uniformity with different linker length BAMFAM-TAM-ddATP using various mutant DNA polymerases.

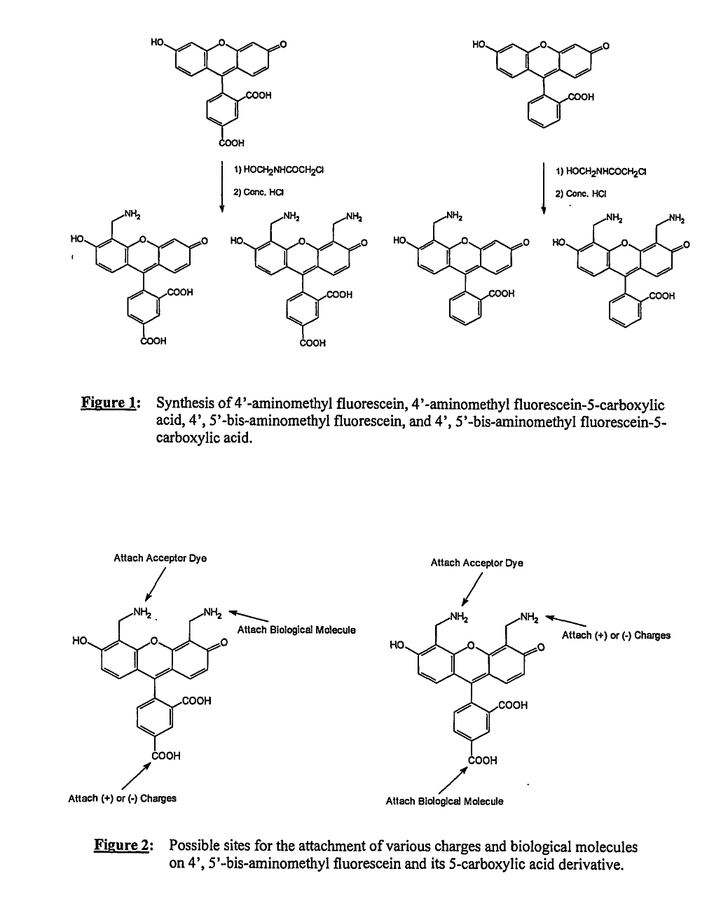

### DETAILED DESCRIPTION OF THE INVENTION

The efficiency of FRET depends on a number of factors. According to Forster's theory (Joseph R. Lakowicz, “Principles of Fluorescence Spectroscopy” 2nd Edition, Chapter 13, Kluwer Academic Plenum Publishers, New York, Boston, Durdrecht, London, Moscow 1999), they are primarily:
1) The overlap of the emission spectrum of the donor and the absorption spectrum of the acceptor,
2) The separation, in distance, between the donor and the acceptor, and,
3) The spatial orientation between the dipoles of the donor and the acceptor.

In practice, the situation is much more complicated. Specific interactions between the donor and the acceptor, may in cases, lead to quenching with very little emission from the acceptor even when the donor emission is completely absent. Furthermore, the extent to which the donor is quenched has very little bearing to what amount of energy being transferred to the acceptor and, hence, its emission to be observed. We have developed a mathematical treatment to describe the practical ET process involved in these dye-terminators. In such a mathematical treatment, three experimentally measurable parameters are of paramount importance:
PQEQ (Percentage of quenching of the donor) which is defined as; PQEQ=(1−Emission_{donor in the donor/acceptor pair}/Emission_{same amount of donor in the absence of the acceptor})×100%, 1)
PAEE (Percentage Acceptor Emission Efficiency): PEAA=(Emission efficiency of the acceptor in the donor acceptor pair)/(Emission efficiency of the acceptor without the donor)×100%, and 2)
PET (Percentage Energy Transfer), PET=Quantum yield of the donor×(Emission efficiency of acceptor when excited at the donor excitation wavelength)/(Emission efficiency of the donor in the absence of the acceptor).

PET, as defined, actually becomes the quantum yield of the donor/acceptor pair when excited at the donor excitation wavelength and the emission measured at the acceptor emission wavelength.

The above methodology can even be extended to ET assemblies consisting of one donor and more than two acceptors. Furthermore, from these numbers, a flow diagram can be constructed to show the photon flow throughout the ET process. As an example, the numbers for the set of four ET dye-terminators (Kumar et.al. PCT WO 00/13026; Nampalli et.al. Tetrahedron Letters 2000, 41, 8867) used in current DNA sequencing reactions are given in Table 1.

TABLE 1
(All measurements in 1 × TBE + 8 M urea buffer, 488 nm excitation)

| Compound* | PQEQ | PAEE | PET |
|---|---|---|---|
| FAM-R110-11-ddGTP (I) | 92% | Not measurable** | 8% |
| FAM-R6G-11-ddUTP (II) | 99% | 47% | 26% |
| FAM-TAM-11ddATP (III) | 98% | 48% | 16% |
| FAM-ROX-11ddCTP (IV) | 99% | 35% | 19% |

*The structures of these compounds are given in below.
**The emissions from FAM and R110 are not resolvable.

The molecular structures of the compounds listed in table one are given below.

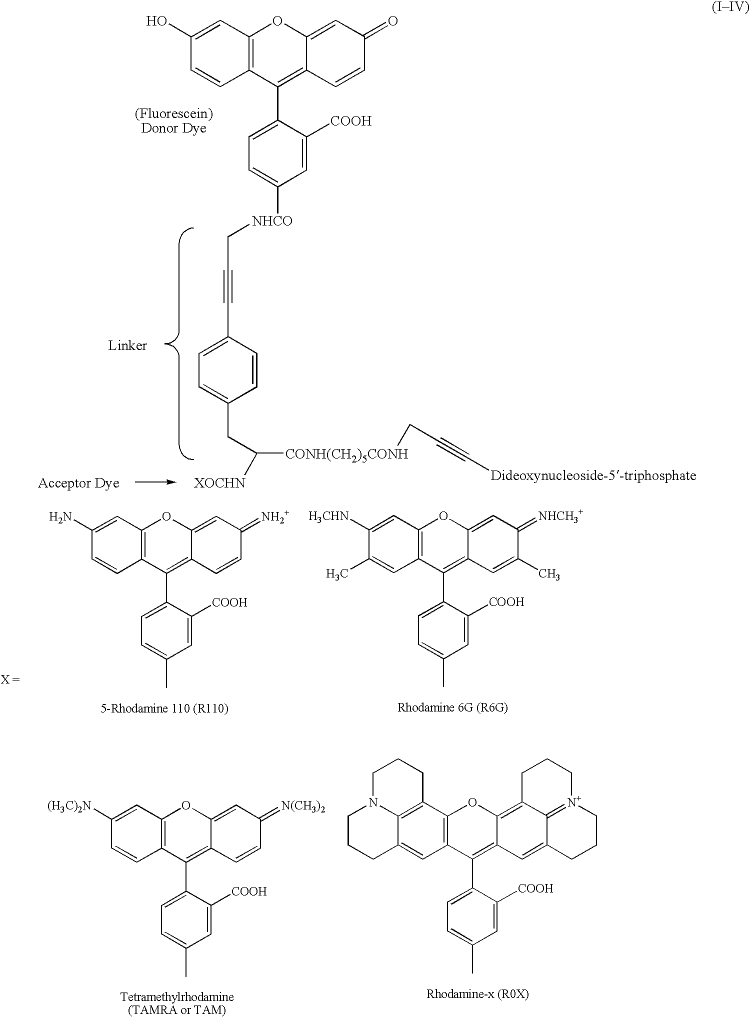

As an illustration, a photon flow diagram can be constructed using the compound (IV) as an example.

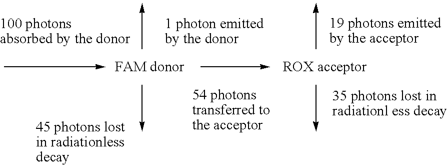

The construction of the diagram is relatively simple. Since PQEQ for the compound (IV) is equal to 99%, only one photon in 100 photon absorbed by the donor (FAM) is re-emitted by the donor. We have PET equals to 0.19, it means that for the 100 photons absorbed by the donor, 19 photons are emitted by the acceptor (ROX). Since the PAEE is 0.35, and assuming that the quantum yield of ROX in its free state is 1.0 relative to FAM, we need the input of 19/0.35 or 54 photons to have 19 photons emitted by ROX acceptor. It follows that the number of photons lost by the acceptor (ROX) in processes other than fluorescence must be 35 (54-19). Then, from the conservation of photons, the number of photons lost in radiationless processes from the donor FAM should be 45 (100-1-54).

During our extensive search for improved brightness of dye terminator sets to that listed in Table One, we discovered a novel type of ET dyes based on 4′,5′-bis-aminomethyl fluorescein (BAM-FAM) and its 5-substituted derivatives (5-carboxy-BAM-FAM). BAM-FAM serves both as the donor fluorophore as well as structural linkages to which an acceptor fluorophore and a modified nucleic acid base can be attached to form dye terminators superior in brightness and other desirable characteristics. 5-carboxy-BAM-FAM even allows one more site for attachment, making trifluor dye-terminator (with one donor and two acceptors, same or different, and a nucleic acid base) architecture possible.

Thus this invention provides energy transfer dyes of the formula: 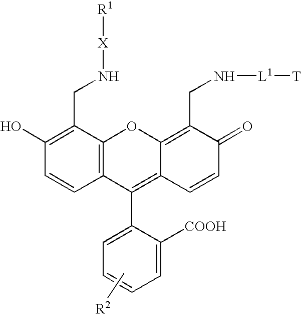
wherein:
R¹ is an acceptor dye selected from the group consisting of xanthine dyes, rhodamine dyes and cyanine dyes and wherein R¹ is capable of accepting energy from a fluorescein donor chromophore;
L¹ is a linker chain containing from 2˜50 linked atoms selected from carbon oxygen, nitrogen, sulphur and phosphorus atoms;
   wherein
   said chain optionally includes one or more positively or negatively charged groups or one or more unsaturated groups selected from —CR═CR— and —C≡C—, wherein R represents hydrogen or a C₁-C₄ alkyl;
   or
   said chain optionally includes one or more positively or negatively charged groups and one or more unsaturated groups selected from —CR═CR— and —C≡C—, wherein R represents hydrogen or a C₁-C₄ alkyl;
R² represents H, COOR or CH₂OR; or a chain L² containing from 2-30 linked atoms selected from carbon oxygen, nitrogen, sulphur and phosphorus atoms;
   wherein
   said chain optionally includes one or more positively or negatively charged groups or one or more unsaturated groups selected from —CR═CR— and —C≡C—, wherein R is hereinbefore defined;
   or
   said chain optionally includes one or more positively or negatively charged groups and one or more unsaturated groups selected from —CR═CR— and —C≡C—, wherein R is hereinbefore defined;
X is selected from 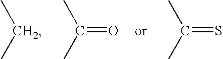
   and
T is a Biological Molecule.

Another embodiment provides an energy transfer dye of the formula 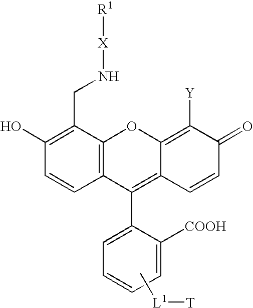
wherein:
R¹ is an acceptor dye selected from the group consisting of xanthine dyes, rhodamine dyes and cyanine dyes and wherein R¹ is capable of accepting energy from a fluorescein donor chromophore;
L¹ is a linker chain containing from 2-50 linked atoms selected from carbon oxygen, nitrogen, sulphur and phosphorus atoms;
   wherein
   said chain optionally includes one or more positively or negatively charged groups or one or more unsaturated groups selected from —CR═CR— and —C≡C—, wherein R represents hydrogen or a C₁-C₄ alkyl;
   or
   said chain optionally includes one or more positively or negatively charged groups and one or more unsaturated groups selected from —CR═CR— and —C≡C—, wherein R represents hydrogen or a C₁-C₄ alkyl;
Y represents H, CH₂NH₂, CH₂NHCOR or CH₂OR wherein R is defined as above; or a chain L² containing from 2-30 linked atoms selected from carbon oxygen, nitrogen, sulphur and phosphorus atoms;
   wherein
   said chain optionally includes one or more positively or negatively charged groups or one or more unsaturated groups selected from —CR═CR— and —C≡C—, wherein R is hereinbefore defined;
   or
   said chain optionally includes one or more positively or negatively charged groups and one or more unsaturated groups selected from —CR═CR— and —C≡C—, wherein R is hereinbefore defined;
X is selected from 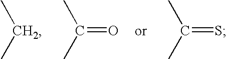
   and
T is a Biological Molecule.

In another aspect, the invention relates to a method for determining the sequence of a nucleic acid, said method comprising:
a) providing a sample of said nucleic acid to be sequenced, a primer nucleic acid sequence which is complementary to at least a part of said nucleic acid to be sequenced, a supply of deoxynucleotides and at least one dideoxynucleotide according to claim 8, for terminating the sequencing reaction, and a polymerase;
b) performing nucleic acid chain extension and chain termination reactions;
c) separating the oligonucleotide fragments according to size.

Additionally, the invention relates to a kit for DNA sequencing comprising at least one compounds described above and wherein, the target molecule is a biological molecule as described above, wherein T is a dideoxynucleoside-5′-triphosphate selected from the group consisting of 2′,3′-dideoxycytidine-5′-triphosphate, 2′,3′-dideoxythymidine-5′-triphosphate, 2′,3′-dideoxyuridine-5′-triphosphate, 2′,3′-dideoxyadenosine-5′-triphosphate, 2′,3′-dideoxyguanosine-5′-triphosphate and 2′,3′-dideoxyinosine-5′-triphosphate, 2′,3′-dideoxy-7-deazaadenosine-5′-triphosphate, 2′,3′-dideoxy-7-deazaguanosine-5′-triphosphate, 2′,3′-dideoxy-7-deazainosine-5′-triphosphate, and 3′-fluoro, 3′-azido, 3′-amino, or 3′-thio derivatives of the above.

As mentioned before, suitable R¹ acceptor dyes are dyes capable of accepting energy from the fluorescein donor chromophore, which forms part of the energy transfer compound of the present invention. For example, suitable acceptor dyes may be selected from the group consisting of xanthine dyes (e.g., fluorescein, naphthofluorescein, rhodol, and derivatives of the aforementioned), rhodamine dyes (e.g., Rhodamine 110, REG, TAMRA, ROX, Texas Red, etc) and cyanine dyes (e.g., Cy3.5, Cy5, Cy5.5, Cy7, etc.) wherein R¹ is capable of accepting energy from a fluorescein donor chromophore;

Some specific suitable acceptor dyes are as follows: fluorescein, naphthofluorescein, rhodol, 5-carboxyrhodamine, 6-carboxyrhodamine, 5-carboxyrhodamine-6-G, 6-carboxyrhodamine-6-G,N,N,N′,N′-tetramethyl-5-carboxyrhodamine, N,N,N′,N′-tetramethyl-6-carboxyrhodamine, 5-carboxy-X-rhodamine, 6-carboxy-X-rhodamine, Cy3(3-(ε-carboxypentyl)-1′-ethyl-3,3,3′,3′-tetramethyl-5,5′-disulphonato-carbocyanine), Cy3.5(3-(ε-carboxypentyl)-1′-ethyl-3,3,3′,3′-tetramethyl-4,5,4′,5′-(1,3-disulphonato)dibenzo-carbocyanine, Cy5(3-(ε-carboxypentyl)-1′-ethyl-3,3,3′,3′-tetramethyl-5,5′-disulphonato)dibenzo-dicarbocyanine, Cy5.5((3-(ε-carboxypentyl)-1′-ethyl-3,3,3′,3′-tetramethyl-4,5,4′,5′-(1,3-disulphonato)dibenzo-dicarbocyanine, and Cy7((3-(ε-carboxypentyl)-1′-ethyl-3,3,3′,3′-tetramethyl-5,5′-(1,3-disulphonato)tricarbocyanine. However, other classes of dyes may also be used as acceptor dyes.

The efficiency of Energy Transfer (ET) is dependent on the distance and the orientation of the donor and acceptor dyes. The connecting linkage in compound (III) of Table One, between the donor, fluorescein (FAM), and the acceptor, tetramethylrhodamine (TAMRA) has a separation of eleven bond lengths between them. However, the connection can be as little as three bond lengths, as shown in BAM-FAM based compound such as (V).

Actually, Lee et.al (Nucleic Acids Research 1997, 25(14), 2816-2822) studied the ET dye “bifluor-1” (V). Nevertheless, these authors concluded at the time, “However, bifluor-1 was no brighter than tetramethylrhodamine alone”. In our investigation, greatly improved ET has been observed in ET terminators derived from either compound (V) or 4′,5′-bis-aminomethylfluorescein (VI) and its 5 or 6-carboxyl derivative (VII).

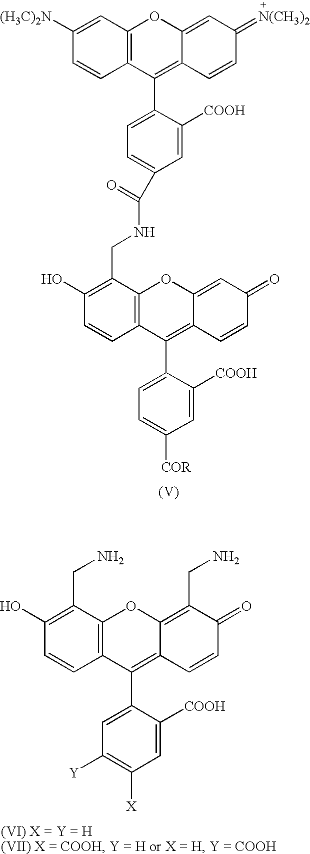

The syntheses of 4′-aminomethyl fluorescein-5-carboxylic acid or 4′-aminomethyl fluorescein-6-carboxylic acid and 4′,5′-bis-aminomethyl fluorescein or 4′,5′-bis-aminomethylfluorescein-5-carboxylic acid and 6-carboxylic acid were undertaken as shown in FIG. 1. Thus, fluorescein or fluorescein-5 or 6-carboxylic acid was reacted with 2-chloro-n-(hydroxymethyl)-acetamide in a sulfuric acid solution and then hydrolyze with conc. HCl to give the corresponding aminomethyl derivatives.

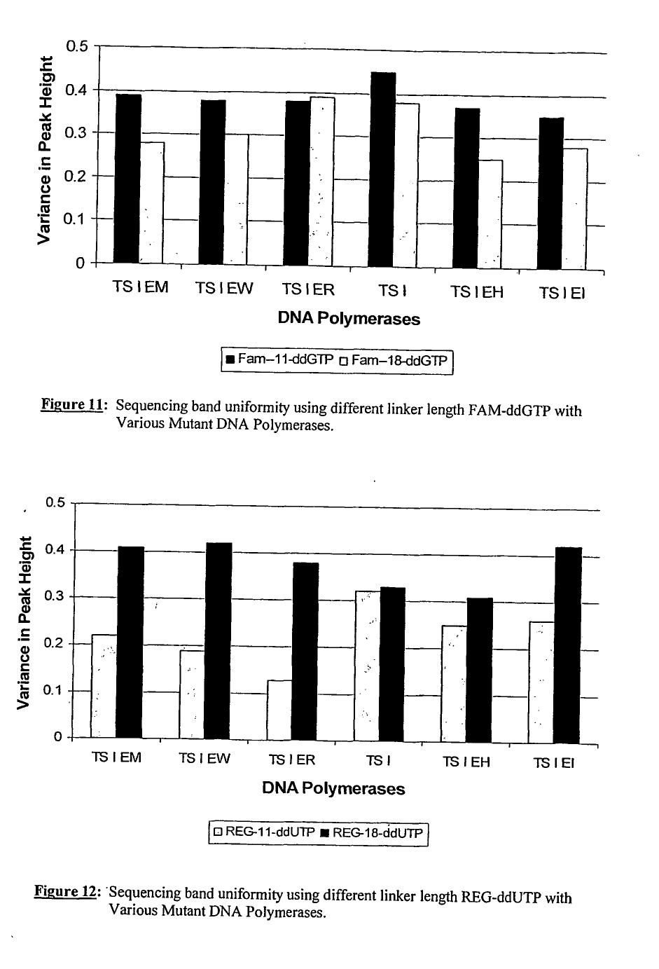

A number of single dye labeled terminators with different linkers between the dye and the dideoxynucleoside-5′-triphosphates were synthesized and their brightness (PET) was measured by excitation at 488 nm. The selection of each terminator in the method of the present invention was made on the basis of terminator reactivity with the DNA polymerase, band uniformity and resolution across the entire sequence (e.g., see FIGS. 11 and 12). The brightness of single dye labeled terminators was also compared with energy transfer dye labeled terminators of the present invention and terminators previously disclosed (see Table 1(supra), and Table 2 (infra)).

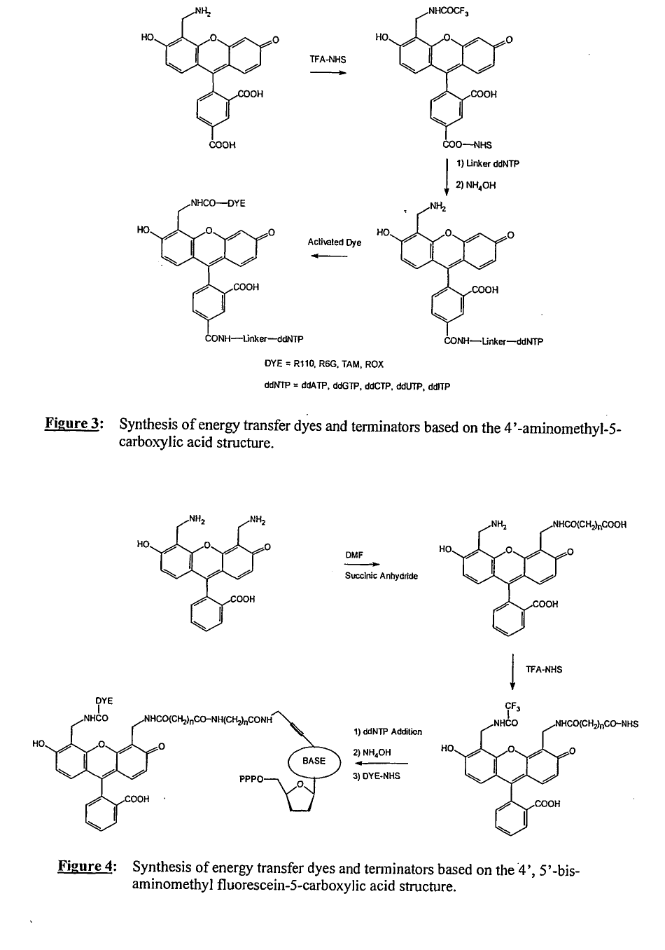

The synthesis of mono-aminomethyl fluorescein derived terminators was undertaken as shown in FIG. 3. Thus, 4′-aminomethyl fluorescein-5-carboxylic acid was reacted with trifluoroacetamido-N-hydroxy-succinarnide (TFA-NHS) to give acid activation and amino protection in one single step The active ester was reacted with appropriately linked propargylamino-ddNTP and hydrolyzed with ammonium hydroxide to provide free amino compound which was reacted with appropriate dye-NHS ester to provide energy transfer dye terminators.

The synthesis of 4′,5′-bis-aminomethyl fluorescein derived terminators was undertaken as disclosed in FIG. 4. In this case, the bis-aminomethyl fluorescein was monoacylated with succinic anhydride followed by reaction with TFA-NHS to provide 4′(5′)-trifluoroacetamidofluorescein-5′(4′)-succinylamidomethyl-NHS ester. The next two steps were carried out essentially the same way as described above for the synthesis of 4′-aminomethyl fluorescein derived energy transfer terminators.

The energy transfer efficiency (PET) was measured for all the single dye labeled dideoxynucleoside-5′-triphosphates (terminators) and the energy transfer terminators synthesized in this invention. All the dye terminators were excited at 488 nm and emission was measured at their respective emission wavelengths. The results are provided in Table 2.

TABLE 2
(All measurements in 1 × TBE + 8 M urea buffer, 488 nm excitation)

| Dye terminator | PQEQ | PAEE | PET |
|---|---|---|---|
| FAM-18-ddGTP (IX) | | | 38* |
| R110-18-ddGTP (X) | | | 28* |
| REG-11-ddATP (XI) | | | 28* |
| TAM-11-ddATP (XII) | | | 1* |
| ROX-11-ddCTP (XIII) | | | 1* |
| AMFAM-R110-11-ddGTP (XIV) | 96 | not measurable** | 4 |
| AMFAM-R6G-11-ddUTP (XV) | 68 | 25 | 21 |
| AMFAM-TAM-18-ddATP (XVI) | 99 | 65 | 29 |
| BAMFAM-TAM-22-ddATP (XVII) | 100 | 55 | 25 |
| AMFAM-ROX-11-ddCTP (XVIII) | 98 | 100 | 46 |
| BAMFAM-ROX-11-ddCTP (XIX) | 97 | 95 | 49 |

*For single dye terminators, PET equivalent = quantum yield of the Single dye-terminator x (extinction coefficient at 488 nm)/(extinction coefficient at absorption maximum).
**Emission from the R110 can not be resolved from that of FAM.

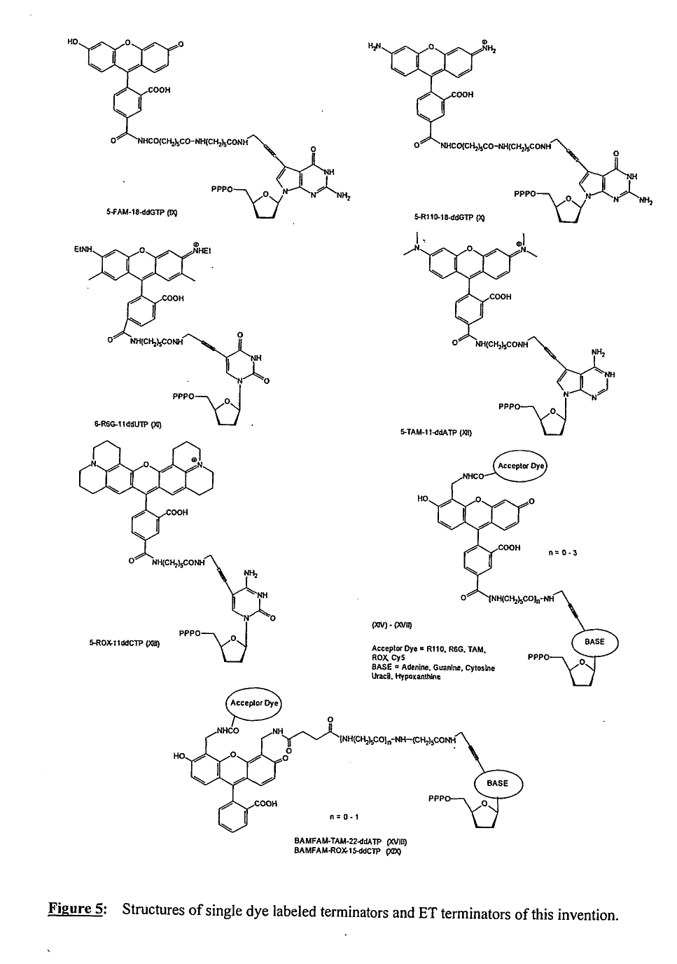

The molecular structures of the dye terminators listed in TABLE TWO are given in the FIG. 5.

It is clear from these PET measurements that the single dye terminators derived from the dyes, FAM, R110, and REG are brighter than the ET constructs derived from FAM-R110 and FAM-REG combinations. The ET dye constructs derived from FAM-TAM and FAM-ROX are brighter than the single dye terminators derived from TAMRA and ROX alone. Contrary to what was reported in literature (Nucleic Acids Research 1997, 25(14), 2816-2822) for the dyes, the ET dye terminator (XVI) is much brighter than the single dye-terminator (XII).

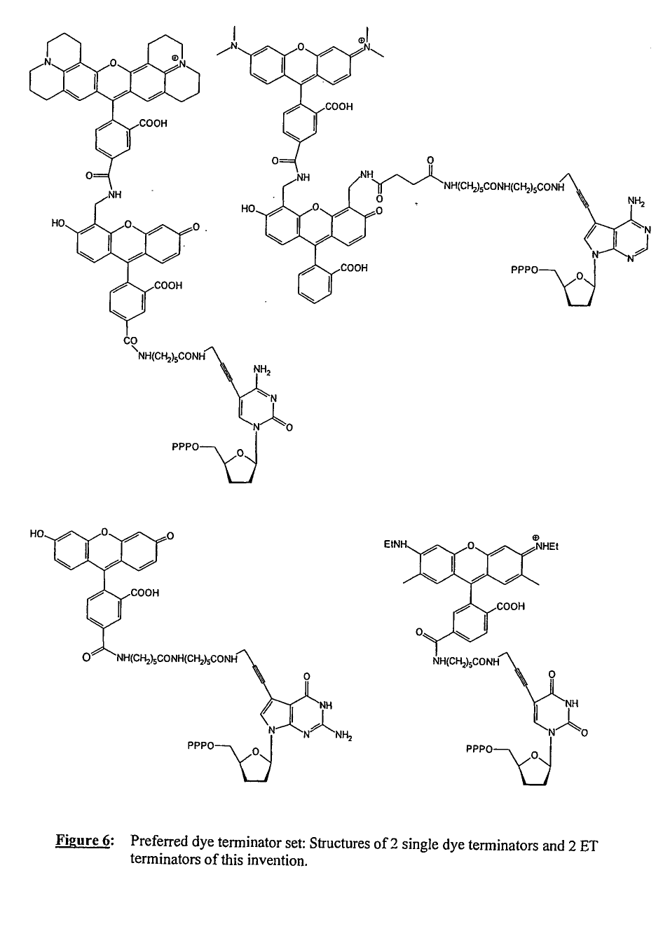

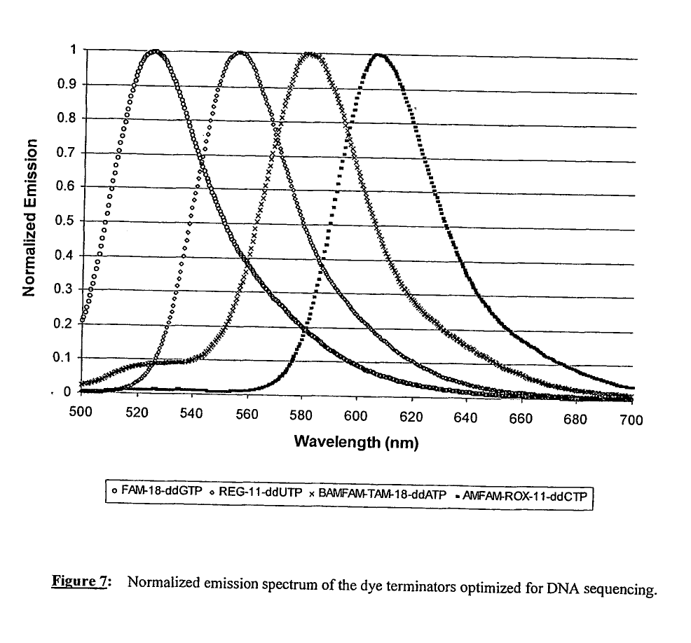

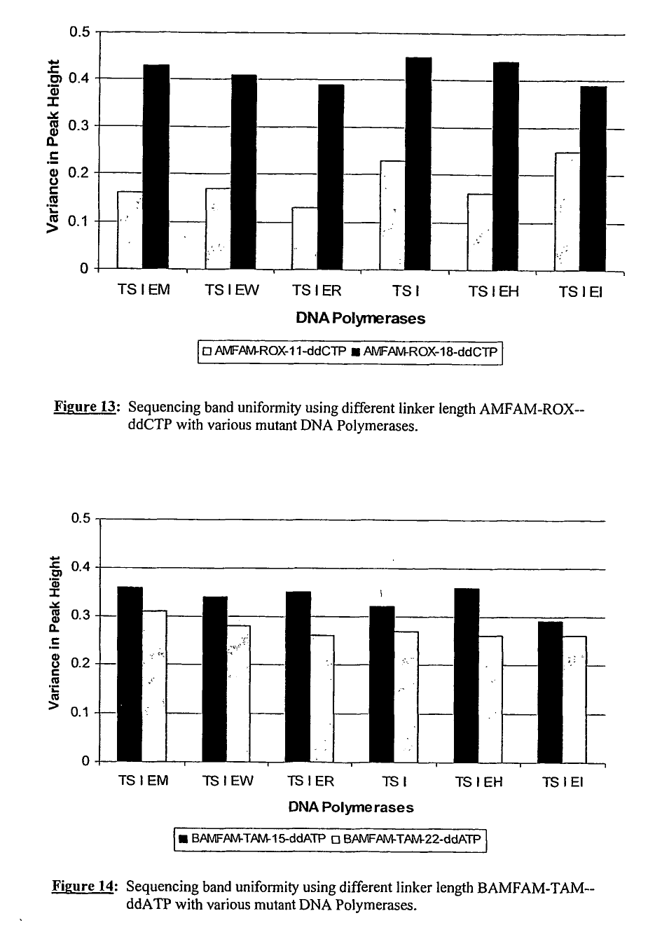

The single dye labeled terminators and energy transfer dyes labeled terminators of this invention (described above) were tested in DNA sequencing reactions using thermostable DNA polymerase. The utility of individual dye labeled terminator was ascertained based on the overall sequence quality, brightness, and uniformity of the bands (FIG. 11-14). Based on these criterions, a new set of dye-terminators was constructed (FIG. 6). The PET of terminators of this new set is given below.

| | PET or | |
|---|---|---|
| Dye-terminator | equivalent | DYEnamic ET set |
| FAM-18-ddGTP (IX), | 38 | 8 (I) |
| R6G-11-ddUTP (XI), | 28 | 26 (II) |
| BAMFAM-TAM-22-ddATP (XVII), | 25 | 16 (III) |
| AMFAM-ROX-11-ddCTP (XVIII) | 46 | 19 (IV) |

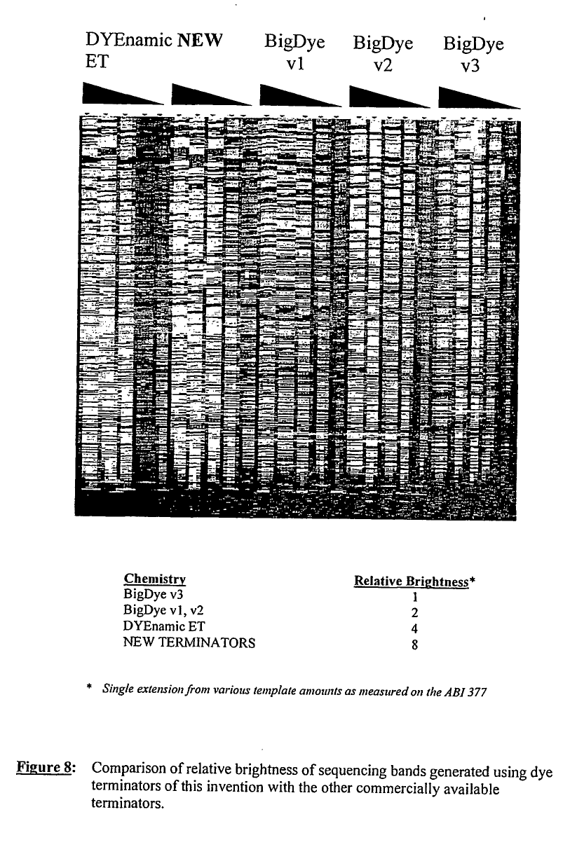

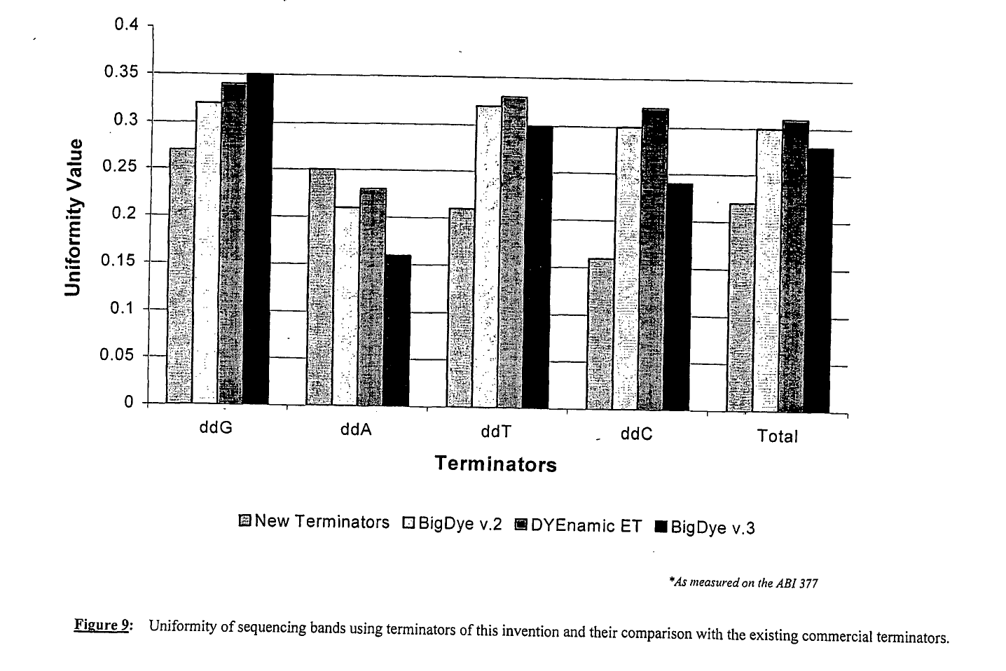

The terminator set of this invention is brighter (FIG. 8) than the current commercial dye terminator sets available from Amersham Biosciences (Piscataway, N.J.) or Applied Biosystems (Foster City, Calif.). This new set, by several measurements is about two-fold brighter than ET terminators listed in TABLE ONE, Four-fold brighter than BigDye™ version 1 and 2 terminators, and about eight-fold brighter than BigDye version 3 terminators (Applied Biosystems (Foster City, Calif.). This increase in brightness means that smaller amounts of template are detectable, fewer cycles may be needed, and sequencing of poor templates is more likely to result in a successful sequence. This new set of dye-terminator has the further advantage of correcting three common artifacts in electropherogram such as fast G's, sloping Ts and weak G's after A's. It is also more uniform in peak heights (FIG. 9).

Compounds such as (VIII), shown with preferred positions for attachment below, furthermore, offer the opportunity of preparing an ET trifluor dye terminator with one donor fluorophore and two acceptors, identical or different to in addition to attached nucleic bases.

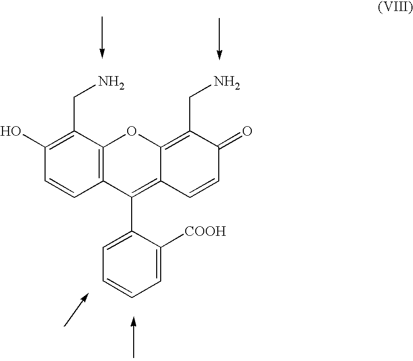

An assembly such as this can extend the emission range of ET dye terminators with the introduction of two different acceptor fluorophores since we can depend on two consecutive energy transfers from the donor. First to the first acceptor fluorophore and, then, from the first acceptor fluorophore to the second while keeping reasonable spectral overlaps among the three fluorophores. Furthermore, there are more than one way to excite the combined fluorophores. One is to excite the fluorescein fluorophore at 488 nm and allow the energy transfer process to generate emission from the longest emitting fluorophore. If we excite both fluorescein and the second fluorophore simultaneously, at 488 nm and the absorption maximum of the second fluorophore, we shall have the energy absorbed by both transferred to the third fluorophore for emission to give the maximum brightness from the third fluorophore

The labeling complexes of the invention are synthesized preferably by covalently linking 4′,5′-bisaminomethyl-fluorescein-5-carboxylic acid to other fluorophores to form energy donor-acceptor complexes. The invention also includes a reagent and a method for making the reagent including incubating the fluorescent water-soluble labeling complex described above with a carrier material. For purposes of bonding the complex and a carrier, the complex may contain a functional group that will react with a reactive group on the carrier to form a covalent bond. Alternatively, the complex may contain a reactive group that will react with a functional group on the carrier to form a covalent bond. The carrier material can be selected from the group consisting of polymer particles, glass beads, cells, antibodies, antigens, proteins, enzymes, nucleotides derivatized to contain one of an amino, sulflhydryl, carbonyl, carboxyl, or hydroxyl groups. As mentioned above, the carrier material may contain the reactive groups and the fluorescent labeling complex of the invention may contain any of the aforementioned functional groups that will react with the reactive group to form covalent bonds.

In an alternative embodiment, the fluorescent complexes of the invention need not have a reactive group when used to noncovalently bind to another compound. For example, the complex may be dissolved, then mixed in an organic solvent with a polymer particle, such as polystyrene then stirred by emulsion polymerization. The solvent is evaporated and the fluorescent dye complex is absorbed into the polystyrene particles.

The invention is further described by reference to the following examples. These examples are provided for illustration purposes only and should not be construed as limiting the appended claims and the scope of the invention. The current invention should encompass any and all variations that become evident from the teachings provided herein.

### EXAMPLES

### 1) Preparation of FAM-18-ddGTP (IX)

A solution of 11-ddGTP (0.1M NaHCO₃/Na₂CO₃, pH 8.5, 60 μmoles, 5 ml) was cooled on an ice/water bath. To the solution was added 5-carboxy-fluorescein-NHS (35 mg, 1 eq.) in DMF (5 ml). The reaction flask was removed from the cooling bath and the reaction mixture was stirred at room temperature for 16 h. The product purified by anion exchange chromatography and HPLC. The product containing fractions were concentrated then lyophilized to yield a yellow solid.

### 2) Synthesis of 4′,5′-bis-aminomethyl fluorescein (BAM-FAM) (FIG. 1)

### a) Preparation of 4′,5′ bis-(2-chloroacetamido)-aminomethyl fluorescein

Fluorescein (3.3 grams) and 2-chloro-n-(hydroxymethyl)-acetamide (5.0 grams) were dissolved in 20 ml of concentrated sulfuric acid. The dark brown solution was stirred at room temperatures for two hours. At this time, ESMS+ indicated that there was no fluorescein left. The product was poured into 200 grams of ice and water and the precipitate was filtered, washed with water, followed by ether and air-dried. NMR of the material, thus, obtained indicated that it was the desired product.

### b) Concentrated hydrochloric acid hydrolysis of 4′,5′-bis-(2-chloroacetamido)-aminomethyl fluorescein to give 4′,5′-bis-aminomethyl fluorescein (BAM-FAM)

The product from the above reaction was suspended in 40 ml of concentrated hydrochloric acid and heated to reflux for 30 minutes. By then, a clear solution was obtained. The product was evaporated down to dryness and the residue recrystalized from Methanol/dichloromethane to give the desired product identified by its NMR and ESMS+

### 3) Synthesis of 4′,5′-bis-aminomethyl fluorescein-5-carboxylic acid (FIG. 1)

Since fluorescein-5-carboxylic acid is only sparingly soluble in concentrated sulfuric acid, a modified procedure was employed

### a) Preparation of 4′,5′-bis-(2-chloroacetamido) aminomethyl fluroscein-5-carboxylic acid

To 20 ml of concentrated sulfuric acid, stirred at room temperatures, was added dipivaloyl-fluorescein-5-carboxylic acid. To the suspension was added, in portions, excess (4 equivalents) of 2-chloro-n-(hydroxymethyl)-acetamide until a clear solution was obtained. More of the starting material (both fluorescein and excess HOCH₂NHCOCH₂Cl) was added until the color of the solution turned from light yellow to brown. The solution was, then, poured onto ice and water mixture. The precipitate, thus obtained was filtered, washed with water and ether. NMR and ESMS+ of the precipitate indicated that it was the desired product.

### b) Concentrated Hydrochloric acid hydrolysis of 4′,5′ bis-(2-chloroacetamido)aminomethyl-fluorescein-5-carboxylic acid to give 4′,5′-bis-aminomehthyl fluroscein-5-carboxylic acid

The product from above, 1.01 grams, was suspended in 20 ml of concentrated hydrochloric acid and 5 ml of 2-methoxyether. The resulting suspension was heated to reflux until it began to clear (ca. two hours). The solution was, then, allowed to cool. After standing at room temperature overnight, the precipitate was filtered, washed with 0.1 N hydrochloric acid followed by ether to give the desired product as shown by its NMR and ESMS+ spectra.

### 4) Preparation of Trifluoroacetamidomethyl-fluorescein NHS ester (TFA-AMFAM-NHS) (FIG. 3)

4′-Aminomethyl-5-carboxyfluorescein (1, 0.49 g, 1.21 μmol) was dried by co-evaporation with dry DMF (15 mL). It was suspended in dry pyridine to which TFA-NHS (1.53 g, 7.26 μmol) was added under argon atmosphere. The reaction mixture was stirred at room temperature for 1 h, diluted with chloroform (125 mL) and washed with water (3×75 mL). Organic layer was dried (sodium sulfate), evaporated and co-evaporated with toluene to give a yellow solid.

### 5) Preparation of 11-ddCTP derivative of TFA-AMFAM-NHS. (FIG. 3)

The product obtained from above (25 mg) was dissolved in DMF (1 ml). To the solution was added either a solution of 11-ddCT? in DMSO (1 mL, 33 μmol) or a solution of 11-ddCTP in pH 8.5 carbonate/bicarbonate buffer (34 μmol). The reaction mixture was stirred at room temperature for 20 h. At such time, 10 mL of concentrated ammonium hydroxide was added. After stirring for 3 h at room temperature, evaporated to a small volume. The crude product was diluted with water (10 mL) and purified by ion exchange column chromatography followed by reverse phase column chromatography.

### 6) Synthesis of AMFAM-ROX-11-ddCTP (XVII). (FIG. 3)

The product (60 μmol) obtained as above was dissolved in dry DMSO (5 mL) to which ROX-NHS ester (60 mg, 60 μmol) was added and the reaction mixture was stirred at room temperature for 20 hrs. The reaction mixture was diluted with water and purified by ion exchange column chromatography followed by reverse phase chromatography.

### 7) Preparation of -AMFAM-TAM-18-ddATP (XVI). (FIG. 3)

In a similar manner as described for the preparation of compound (XVII), conjugation of TFA-AMPAM-NHS with 18-ddATP followed by reaction with TAMRA-NHS ester gave the target compound (XVI).

### 8) Preparation of Cv-5-AMFAM (XX)

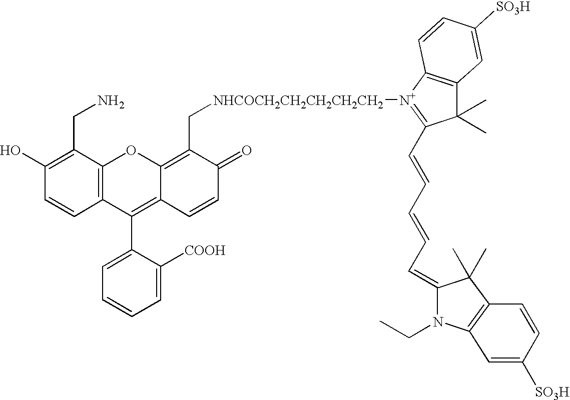

4′-methylamino-fluorescein, 5 mg, was dissolved in 0.5 ml of dry DMF. To the solution was added 20 mg of Cy-5 mono-functional reactive dye in 1.0 ml of sodium bicarbonate-carbonate buffer. After 20 minutes at room temperatures, the solvents were evaporated and the residue chromatographed to give the desired product (XX).

### 9) Synthesis of Cy-5 AMFAM-TAM(XXI)

### a) Preparation of the AMFAM-TAM (XVIa)

4′,5′ bis-aminomethyl-fluorescein, 4 mg, and 5-carboxytetra-methylrhodamine succinimidyl ester, 10 mg, were dissolved in 1 ml of dry dimethylformamide (DMF) with excess diethyl-n-isopropylamine. The reaction was allowed to proceed, at room temperatures, overnight and the product obtained was purified by TLC to give the desired (XVIa).

### b) Preparation of the Cy-5 AMFAM-TAM (XXI)

The compound (XVIa), obtained above, was dissolved in dry dimethylforamide with excess diethyl-n-isopropylamine added. To the solution, was added a slight excess of Cy-5 mono-functional NHS ester in carbonate/bicarbonate buffer. After the reaction was over, as shown by the disappearance of the starting (BA) on thin layer chromatogram (TLC), the solvent(s) was evaporated to dryness and the residue chromatographed on C18 reversed phase TLC plate to give the desired product (XXI).

### c) 4′(5′)-Trifluoroacetamidofluorescein-(4′)5′-succinylamidomethyl-NHS ester (FIG. 4)

Bis-aminomethylfluorescein (0.14 g, 0.3 umol) was dried by co-evaporation with dry DMF (10 mL). Then it was dissolved in a mixture of DMF (8 mL) and pyridine (4 mL) to which succinic anhydride (19 mg) was added. After stirring at room temperature for 2 h an additional 7 mg of succinic anhydride was added. The reaction was continued for 2 more hours and the solvents were evaporated to dryness. The crude product contained some starting material, mono succinyl derivative and disuccinyl derivative. The mono and di succinyl derivatives were separated by silica gel column chromatography utilizing 10-30% methanol-dichloromethane containing 0.5% formic acid. The appropriate fractions containing mono succinyl derivative was collected and evaporated to dryness.

The mono succinyl compound (50 mg) was dried by co-evaporation with dry pyridine (2×10 mL), then it was dissolved in a mixture of pyridine (4 mL) and dichloromethane (4 mL) to which TFA-NHS (150 mg) was added. After stirring at room temperature for an hour, the reaction mixture was diluted with dichloromethane (100 mL) and washed with water (2×50 mL). Organic layer was dried (sodium sulfate) evaporated and the residue was co-evaporated with toluene to give 50 mg of a light yellow solid. MS: Calcd.683.24. Found: 682.23 (M-H)⁻

Conjugation with ddNTP derivatives and conversion to ET-Terminators.(FIG. 4).

To a solution of ddNTP (25 umol) in pH 8.5 carbonate/bicarbonate buffer (0.5 mL) was added a solution of compound 8 (25 mg) in DMF (0.4 mL). The reaction mixture was stirred at room temperature overnight and 20 mL of conc. ammonium hydroxide was added. After 3 h, the reaction mixture was evaporated to a small volume, diluted with water and purified by ion exchange column chromatography. The fractions containing the pure product were evaporated and the residue was co-evaporated with methanol (3×50 mL). Then it was dissolved in DMSO (2 mL) to which dye-NHS ester (30 umol, ROX, TAMRA or R6G) was added and the mixture was stirred at room temperature for 20 h. The reaction mixture was diluted with water and purified by ion exchange column chromatography followed by reverse phase column chromatography.

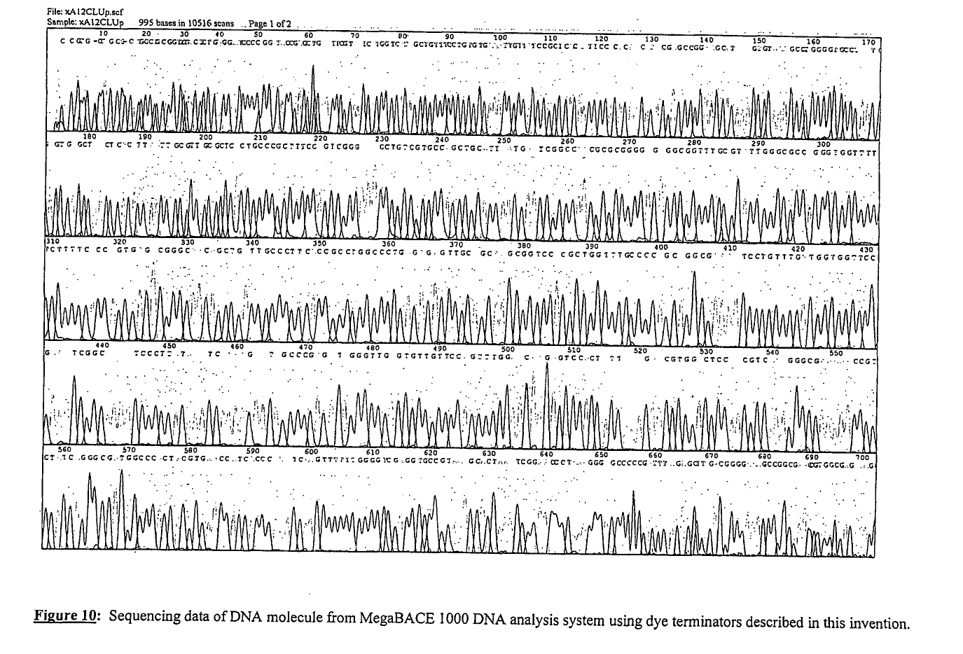

### 10) Sequencing DNA (FIG. 10) Using the Compounds labeled as compounds “IX, XI, XVII and XVIII in Table 2 and FIG. 6

A sequence of M13mp18 template DNA was generated using standard “−40” primer. The reaction mixture (20 μl) contained 200 μM each of dATP, dCTP, and dTTP, 1000 μM dITP, 160 nM FAM-18-ddGTP, 125 nM R6G-11-ddUTP, 95 nM BAMFAM-TAM-22-ddATP, 60 nM AMFAM-ROX-11-ddCTP, 2 pmol −40 primer, 200 ng M13mp18 DNA, 20 units of Thermo Sequenase or other mutated DNA polymerase (Amersham Biosciences), 0.0008 units Thermoplasma acidophilum inorganic pyrophosphatase, 50 mM Tris-HCl pH 8.5, 35 mM KCl and 5 mM MgCl₂.

The reaction mixture was incubated in a thermal cycler for 25 cycles of 95° C., 20 Sec; 50° C., 30 Sec., and 60° C., 120 Sec. After cycling, the reaction products were precipitated with ethanol using standard procedures, washed and resuspended in formamide loading buffer. The sample was loaded on an Applied Biosystems model 377 instrument or MegaBACE 1000 (Amersham Biosciences) and results were analyzed using standard software methods.

Those skilled in the art having the benefit of the teachings of the present invention as set forth above, can effect numerous modifications thereto. These modifications are to be construed as being encompassed within the scope of the present invention as set forth in the appended claims.

1. A compound of the formula: 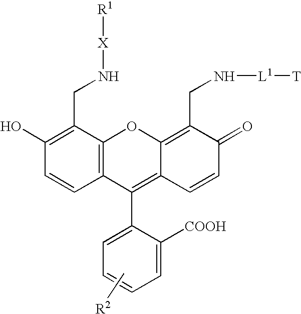
wherein:
R¹ is an acceptor dye selected from the group consisting of xanthine dyes, rhodamine dyes and cyanine dyes and wherein R¹ is capable of accepting energy from a fluorescein donor chromophore;
L¹ is a linker chain containing from 2-50 linked atoms selected from carbon oxygen, nitrogen, sulphur and phosphorus atoms;
wherein
said chain optionally includes one or more positively or negatively charged groups or one or more unsaturated groups selected from —CR═CR— and
—C≡C—, wherein R represents hydrogen or a C₁-C₄ alkyl;
or
said chain optionally includes one or more positively or negatively charged groups and one or more unsaturated groups selected from —CR═CR— and
—C≡C—, wherein R represents hydrogen or a C₁-C₄ alkyl;
R² represents H, COOR or CH₂OR; or a chain L² containing from 2-30 linked atoms selected from carbon oxygen, nitrogen, sulphur and phosphorus atoms;
wherein
said chain optionally includes one or more positively or negatively charged groups or one or more unsaturated groups selected from —CR═CR— and
—C≡C—, wherein R is defined as above;
or
said chain optionally includes one or more positively or negatively charged groups and one or more unsaturated groups selected from —CR═CR— and
—C≡C—, wherein R is defined as above;
X is selected from 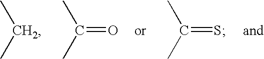
T is a Biological Molecule.

2. A compound of the formula: 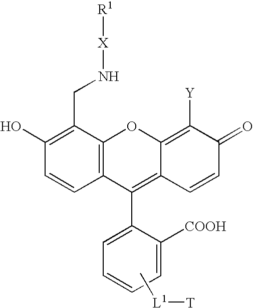
wherein:
R¹ is an acceptor dye selected from the group consisting of xanthine dyes, rhodamine dyes and cyanine dyes and wherein R¹ is capable of accepting energy from a fluorescein donor chromophore;
L¹ is a linker chain containing from 2-50 linked atoms selected from carbon oxygen, nitrogen, sulphur and phosphorus atoms;
wherein
said chain optionally includes one or more positively or negatively charged groups or one or more unsaturated groups selected from —CR═CR— and
—C≡C—, wherein R represents hydrogen or a C₁-C₄ alkyl;
or
said chain optionally includes one or more positively or negatively charged groups and one or more unsaturated groups selected from —CR═CR— and
—C≡C—, wherein R represents hydrogen or a C₁-C₄ alkyl;
Y represents H, CH₂NH₂, CH₂NHCOR wherein R is defined as above, or CH₂OR wherein R is defined as above; or a chain L² containing from 2-30 linked atoms selected from carbon oxygen, nitrogen, sulphur and phosphorus atoms;
wherein
said chain optionally includes one or more positively or negatively charged groups or one or more unsaturated groups selected from —CR═CR— and
—C≡C—, wherein R is hereinbefore defined;
or
said chain optionally includes one or more positively or negatively charged groups and one or more unsaturated groups selected from —CR═CR— and
—C≡C—, wherein R is hereinbefore defined;
X is selected from 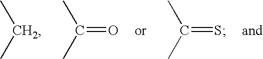
T is a Biological Molecule.

3. The compound of claim 1 or 2 wherein L¹ is a C₂₋₅₀ alkyl chain wherein said chain optionally includes one or more groups selected from —C(O)—, —C(S)—, —NR—, —N⁺R₂—, —O—, —S—, —CR═CR—, —C≡C—, —CO—NR— and 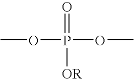
groups, where R is defined as in claim 1 or 2.

4. The compound of claim 1 or 2 wherein L² is a C₂₋₃₀ alkyl chain wherein said chain optionally includes one or more groups selected from —C(O)—, —C(S)—, —NR—, —N⁺R₂—, —O—, —S—, —CR═CR—, —C≡C—, —CO—NR— and 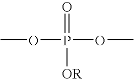
groups, where R is defined as in claim 1 or 2

5. The compound of claim 1 or 2 wherein T is selected from the group consisting of: nucleosides, nucleotides, nucleotide derivatives, oligonucleotides, oligonucleotide derivatives, polynucleotides, polynucleotide derivatives, peptides, proteins, and carbohydrates.

6. The compound of claim 1 or 2 wherein T is selected from the group consisting of ribonucleotides, 2′- or 3′-deoxyribonucleotides, and 2,3′-dideoxyribonucleotides.

7. The compound of claim 1 or 2 wherein T is a nucleoside-5′-mono, di, or triphosphate.

8. The compound of claim 1 or 2 wherein T is a dideoxynucleoside-5′-triphosphate selected from the group consisting of 2′,3′-dideoxycytidine-5′-triphosphate, 2′,3′-dideoxythymidine-5′-triphosphate, 2′,3′-dideoxyuridine-5′-triphosphate, 2′,3′-dideoxyadenosine-5′-triphosphate, 2′,3′-dideoxyguanosine-5′-triphosphate and 2′,3′-dideoxyinosine-5′-triphosphate, 2′,3′-dideoxy-7-deazaadenosine-5′-triphosphate, 2′,3′-dideoxy-7-deazaguanosine-5′-triphosphate, 2′,3′-dideoxy-7-deazainosine-5′-triphosphate, and 3′-fluoro, 3′-azido, 3′-amino, or 3′-thio derivatives of the above.

9. The compound of claim 1 or 2, wherein L¹ is attached to said target molecule at the C-5 position when said target molecule contains a pyrimidine and at the C-7 position when said target molecule contains a 7-deazapurine.

10. The compound of claim 1 or 2, wherein R₁ is selected from the group consisting of: fluorescein, naphthofluorescein, rhodol, 5-carboxyrhodamine, 6-carboxyrhodamine, 5-carboxyrhodamine-6-G, 6-carboxyrhodamine-6-G,N,N,N′,N′-tetramethyl-5-carboxyrhodamine, N,N,N′,N′-tetramethyl-6-carboxyrhodamine, 5-carboxy-X-rhodamine, 6-carboxy-X-rhodamine, Cy3(3-(ε-carboxypentyl)-1′-ethyl-3,3,3′,3′-tetramethyl-5,5′-disulphonato-carbocyanine), Cy3.5(3-(ε-carboxypentyl)-1′-ethyl-3,3,3′,3′-tetramethyl-4,5,4′,5′-(1,3-disulphonato)dibenzo-carbocyanine, Cy5(3-(ε-carboxypentyl)-1′-ethyl-3,3,3′,3′-tetramethyl-5,5′-disulphonato)dibenzo-dicarbocyanine, Cy5.5((3-(ε-carboxypentyl)-1′-ethyl-3,3,3′,3′-tetramethyl-4,5,4′,5′-(1,3-disulphonato)dibenzo-dicarbocyanine, and Cy7((3-(ε-carboxypentyl)-1′-ethyl-3,3,3′,3′-tetramethyl-5,5′-(1,3-disulphonato)tricarbocyanine.

11. A compound of the formula:

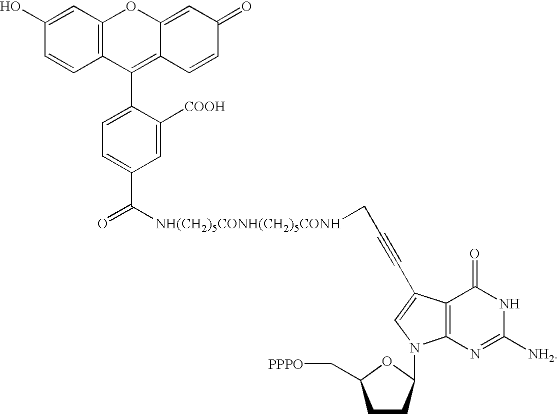

12. A compound of the formula:

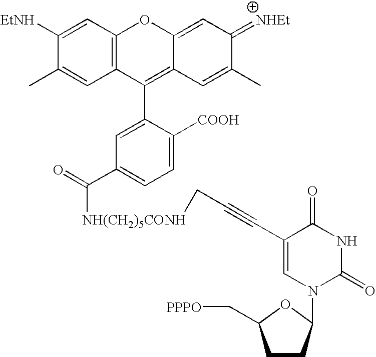

13. A compound of the formula:

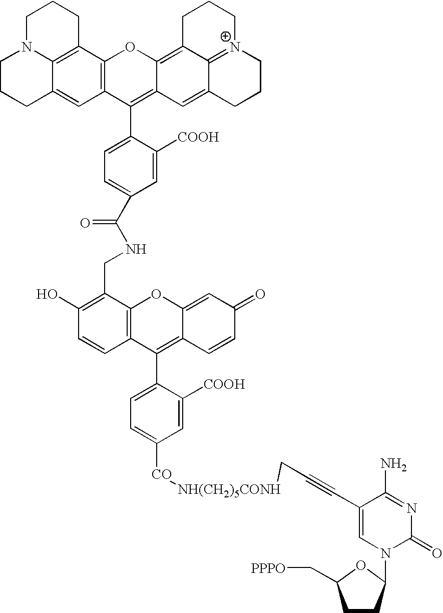

14. A compound of the formula:

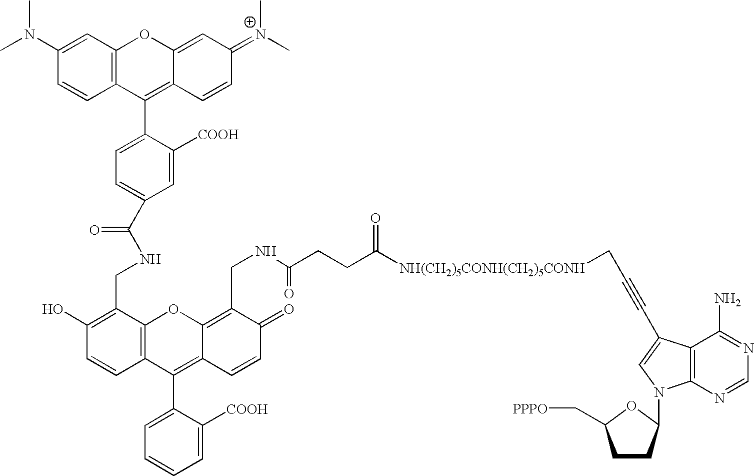

15. A deoxyribonucleic acid sequence comprising the nucleoside portion of one or more compounds according to any one of claims 11-14.

16. A method for determining the sequence of a nucleic acid, comprising:
a) providing a sample of said nucleic acid to be sequenced, a primer nucleic acid sequence which is complementary to at least a part of said nucleic acid to be sequenced, a supply of deoxynucleotides and at least one dideoxynucleotide according to claim 8, for terminating the sequencing reaction, and a polymerase;
b) performing nucleic acid chain extension and chain termination reactions; and
c) separating the oligonucleotide fragments according to size.

17. A method for determining the sequence of a nucleic acid, comprising:
a) providing a sample of said nucleic acid to be sequenced, a primer nucleic acid sequence which is complementary to at least a part of said nucleic acid to be sequenced, a supply of deoxynucleotides and at least one dideoxynucleotide according to any one of claims 11-14, for terminating the sequencing reaction, and a polymerase;
b) performing nucleic acid chain extension and chain termination reactions; and
c) separating the oligonucleotide fragments according to size.

18. A kit for DNA sequencing comprising at least one compound according to claim 7.

19. The kit of claim 18, further comprising a DNA polymerase.
